(19) United States
(12) Patent Application Publication
(10) Pub. No.: US 2006/0222622 A1
(43) Pub. Date: Oct. 5, 2006

(54) Methods and compositions for preventing and treating radiation-induced skin reactions

(76) Inventor: Marie-Pierre Faure, Ville St. Laurent (CA)

Correspondence Address: KIRKPATRICK & LOCKHART NICHOLSON GRAHAM LLP, STATE STREET FINANCIAL CENTER, ONE LINCOLN STREET, BOSTON, MA, 02111-2950, US

(21) Appl. No.: 11/397,652

(22) Filed: Apr. 4, 2006

Related U.S. Application Data
(60) Provisional application No. 60/667,986, filed on Apr. 4, 2005.

(51) Int. Cl. A61K 38/16; A61K 31/765

(52) U.S. Cl. 424/78.27; 514/2; 424/78.38

(57) ABSTRACT

Methods for preventing and treating radiation-induced skin reactions are disclosed. The methods generally include applying one or more hydrogel compositions to a topical site before, during, and/or after exposure to radiation. The hydrogel compositions generally include one or more electrolytes. In some embodiments, the hydrogel compositions can include a protein component and a biocompatible polymer component, where the protein component can be covalently crosslinked by the biocompatible polymer component. The invention further provides methods of preparing such hydrogel compositions.

This application claims the benefit of the filing date of U.S. Provisional Patent Application Ser. No. 60/667,986, filed on Apr. 4, 2005, the entire disclosure of which is incorporated by reference herein for all purposes.

### TECHNICAL FIELD

The invention generally relates to methods for the prevention and treatment of adverse skin reactions resulting from radiation therapy. More specifically, the present invention relates to methods for preventing, reducing, treating, and/or delaying an adverse skin reaction due to ionizing radiation by applying to a topical site one or more hydrogel compositions before, during, and/or after exposing the topical site to such radiation. The invention further provides hydrogel compositions that are useful for the practice of the methods of the invention.

### BACKGROUND

Radiation therapy is a well-established treatment of malignant tumors. Typical regimens include daily (e.g., five times per week) exposure of the tumor to ionizing radiation for five or more weeks with a cumulative ionizing radiation dosage of about 45-80 Gray (Gy). While itself painless, radiation therapy often causes both acute and chronic side effects, particularly with higher doses. One of these side effects is a condition known as radiation dermatitis, the symptoms of which include various skin damage ranging from mild erythema and soreness to moist desquamation and ulceration. The worst cases of radiation dermatitis tend to occur in areas where there are natural folds in the skin, e.g., behind the ear, in the neck area, and underneath the female breast. Patients receiving a combination of radiation therapy and chemotherapy to treat cancer also are at a higher risk of developing severe adverse skin reactions, because the addition of chemotherapy tends to exacerbate radiation side effects.

Acute radiation-induced skin reactions can arise because ionizing radiation has sufficient energy to break chemical bonds thereby degrading and destroying tissues. Ionizing radiation has a particularly adverse effect on rapidly dividing cells, including the basal cells found at the base of the epidermis. Basal cells compensate for cell loss at the surface of the epidermis by rapidly dividing to provide a renewed cell population. When a basal cell divides, two cells are formed. One of these cells begins the progressive process of terminal differentiation into mature, dead, keratinized or cornified cells. From the outermost layer of the epidermis, cornified cells detach and desquamate. Since the average turnover time of the entire epidermis is about 3-4 weeks, acute radiation-induced skin reactions arising from damage to basal cells is typically observed approximately 3-5 weeks after initiation of radiation treatment and can continue for approximately 4 weeks after cessation of the regimen.

Such radiation-induced skin reactions not only are a cause for considerable discomfort and distress to the patients, they also can affect the effectiveness of the radiation treatment. While the degree of skin injury depends on a large number of factors such as the site of irradiation on the patient, the source of radiation, the dose, the treatment schedule and the individual sensitivity of the patient, it is estimated that approximately one-third of patients undergoing radiation therapy for tumors will experience an interruption in a therapy schedule, typically of about 1-3 weeks, to permit the skin to heal sufficiently to resume the treatment. Because maintenance of a daily radiation schedule is clinically significant in the effective treatment of the tumor, interruption of treatment for any significant period of time is highly undesirable.

Ionizing radiation also may cause skin damage, such as atrophy, thinning of the skin, telangiectasia, altered pigmentation, fibrosis, ulceration, necrosis and carcinogenesis, six or more months after termination of radiation treatment. Such skin damage severely jeopardizes the patient's quality of life.

Many attempts have been made to reduce, control or cure radiation dermatitis. U.S. Pat. No. 4,617,187 to Okuyama et al. discloses a method for treating radiation dermatitis by topically applying ubidecarenone. However, ubidecarenone may be toxic and can cause many side effects during the treatment. Other topical formulations including simple moisturizers and steroid creams have been suggested as prophylaxis and/or treatment for radiation dermatitis. Studies on their efficacies are often inconclusive. See e.g., Faithfull et al., SUPPORTIVE CARE IN RADIOTHERAPY, Churchill Livingstone, London (2003). Further, such ointments often are difficult to apply as the patient is required to massage the ointment into the skin until it is substantially absorbed, thereby potentially causing additional pain or skin damage.

Currently, there are no widely accepted protocols and guidelines for the management of radiation-induced skin reactions. It has been described that the management of radiation-induced skin reactions is often ritualistic and preference-led. Thus there remains a need for novel methods and compositions that can effectively prevent and treat adverse skin reactions resulting from radiation therapy.

### SUMMARY OF THE INVENTION

It has been discovered that the application of one or more hydrogel compositions according to the invention before, during, and/or after exposure to ionizing radiation can prevent, treat, and/or delay the onset of radiation dermatitis. As a result, distress and discomfort experienced by a patient during radiation treatment can be greatly minimized. Interruption of radiation treatment also can be prevented.

According to one aspect of the invention, a method of preventing one or more adverse skin reactions resulting from ionizing radiation can include applying a hydrogel composition including one or more electrolytes to a topical site within four hours of a first exposure of the topical site to ionizing radiation. Examples of suitable electrolytes include but are not limited to sodium chloride (NaCl), sodium phosphate (e.g., Na₂HPO₄ and NaH₂PO₄), and a sodium salt of ethylenediaminetetracetic acid (EDTA). The one or more electrolytes should be water-soluble so that the hydrogel composition, when applied, can be hydrated and contain a sufficient amount of electrolyte(s). For example, the hydrogel composition can include a water content of 50% or more by weight, a water content of 60% or more weight, a water content of 70% or more by weight, a water content of 80% or more by weight, a water content of 85% or more by weight, a water content of 90% or more by weight, or a water content of 95% or more by weight. In addition, the hydrogel composition can include about 5-10% by weight of one or more electrolytes, about 2-5% by weight of one or more electrodes, or about 0.5-2% by weight of one or more electrolytes.

The hydrogel composition can include a biocompatible polymer component. The biocompatible polymer component can include one or more natural polymers, synthetic polymers, or combinations thereof. For example, the biocompatible polymer can be a polyalkylene oxide such as polyethylene glycol (PEG) or a derivative of PEG including but not limited to carbonates of polyethylene glycol. The hydrogel composition can further include a protein component covalently crosslinked by the biocompatible polymer. The protein component can include one or more proteins selected from bovine serum albumin, human serum albumin, lactalbumin, ovalbumin, soy albumin, pea albumin, hydrolyzed soy protein, hydrolyzed wheat protein, casein, and any of their combinations.

To prevent one or more skin reactions resulting from ionizing radiation, the hydrogel composition can be preferably applied immediately after a first exposure of the topical site to ionizing radiation. For example, the hydrogel composition can be applied within four hours of a first exposure, within two hours of a first exposure, within one hour of a first exposure, within thirty minutes of a first exposure, within fifteen minutes of a first exposure, within ten minutes of a first exposure, or within five minutes of a first exposure. The hydrogel composition can be continuously applied on the topical site until the next exposure of the topical site to ionizing radiation, typically 24 hours after the first exposure. Because a typical radiation therapy regimen includes five weeks or more of daily exposure to ionizing radiation, a hydrogel composition can be applied daily for five or more consecutive weeks in between exposures to ionizing radiation. In some embodiments, the method includes daily application of a hydrogel composition to the topical site throughout the radiation treatment regimen and for some time thereafter, e.g., for two additional weeks after termination of radiation treatment.

The hydrogel composition can be applied to any anatomical site that can be treated with ionizing radiation, including anatomical sites located in the cervical area, the breast area, the axilla, the abdominal area, the cranial area, the thoracic area, the inguinal area, the perianal area, and the perineum area. It is generally preferred that the hydrogel composition is atraumatic, i.e., non-adherent to the topical site and non-adhesive to the areas surrounding the topical site to be irradiated. For ease of application, the hydrogel composition can include a polymeric backing. The polymeric backing can be attached to the hydrogel composition without an adhesive. Additionally, a mesh may be embedded within the hydrogel composition. In some embodiments, a surface of the hydrogel composition or its backing is attached to a layer of pressure-sensitive adhesive, for example, a biocompatible silicone. The layer of pressure-sensitive adhesive is then applied to the inner side (i.e., the side having contact with a patient's body) of a piece of garment, for example, a bra, thereby securing the hydrogel composition to the topical site without risks of causing trauma due to use of secondary dressings that may adhere to the topical site or surrounding areas. In certain embodiments, the pressure-sensitive adhesive layer can be applied first to the inner side of the piece of garment. In these embodiments, the hydrogel composition can then be deposited on top of the pressure-sensitive adhesive layer.

Another aspect of the invention is generally related to a method of treating one or more adverse skin reactions resulting from ionizing radiation. Such method of treatment can include reducing the degree or accelerating the healing of any skin damage, delaying the onset of adverse skin reactions, and/or alleviating the symptoms of such skin reactions. The method generally includes applying to a topical site an embodiment of the hydrogel compositions described above after exposure of the topical site to ionizing radiation. The topical site can be intact skin or an open wound displaying symptoms of one or more adverse skin reactions. In some embodiments, the method includes daily application of a hydrogel composition to the topical site until all physical signs of skin damage disappear.

The methods of the invention can be useful in preventing and/or treating adverse skin reactions resulting from ionizing radiation. Typical radiation doses are in the range of about 1 Gy to about 80 Gy. Various types of ionizing radiation can be used, including alpha radiation, beta radiation, gamma radiation, x-irradiation, and fluoroscopic radiation. Adverse skin reactions resulting from ionizing radiation can include, but are not limited to, erythema, hyperpigmentation, epilation, edema, dry desquamation, moist desquamation, ulceration, hemorrhage, and necrosis. Such adverse skin reactions can be symptomatic of acute or chronic radiation dermatitis. The methods of the invention also can be useful in preventing and/or alleviating undesirable sensations such as tenderness, burning, itch, and pain.

A further aspect of the invention relates to a method of preparing a hydrogel composition suitable for use in the methods of preventing and/or treating radiation-induced skin reactions described above. The method generally includes reacting a protein component with a bifunctional biocompatible polymer. The protein component can include any of the proteins that have already been described. The bifunctional biocompatible polymer can be a bifunctional polyethylene glycol, and can be selected from a dinitrophenylcarbonyl polyethylene glycol, a dichlorosulfonyl polyethylene glycol, a dichloroacetylsulfonyl polyethylene glycol, a dichlorosulfonyl ethylsulfonyl polyethylene glycol, a diphenylcarbonyl polyethylene glycol, a ditoluenesulfonyl polyethylene glycol, a disuccinimidyl polyethylene glycol, a dimaleimidyl polyethylene glycol, a diisocyanato-polyethylene glycol, and a divinylsulfonamido-polyethylene glycol. The method also can include converting a biocompatible polymer into a bifunctional biocompatible polymer before the reacting step. The converting step can be conducted in solvent or in a solvent-free environment.

The foregoing, and other features and advantages of the invention as well as the invention itself, will be more fully understood from the following figures, description, and claims.

### BRIEF DESCRIPTION OF FIGURES

This patent or application contains at least one drawing executed in color. Copies of this patent or patent application publication with color drawing(s) will be provided by the United States Patent and Trademark Office upon request and payment of the necessary fee.

A skilled artisan will understand that the drawings described below are for illustration purposes only and are not necessarily to scale. The drawings are not intended to limit the scope of the present teachings in any way.

FIG. 1 is a top perspective view of an embodiment of a schematic of a hydrogel composition of the invention deposited on a pressure-sensitive adhesive layer.

FIGS. 2a-c are photographs of the skin fold area underneath the irradiated breast of a patient receiving preventive treatment with an embodiment of hydrogel compositions of the invention. FIG. 2a was taken before a first exposure of the area to radiation, while FIGS. 2b and 2c were taken at week 5 and week 7, respectively.

FIGS. 3a-d are photographs of the skin fold area underneath the irradiated breast of a patient receiving curative treatment with an embodiment of hydrogel compositions of the invention. FIG. 3a was taken before a first exposure of the area to radiation, while FIGS. 3b, 3c and 3d were taken at week 5, week 8 and week 9, respectively.

FIGS. 4a-c are photographs of the skin fold area underneath the irradiated breast of a patient receiving curative treatment with a commercially available trolamine ointment. FIG. 4a was taken before a first exposure of the area to radiation, while FIGS. 4b and 4c were taken at week 5 and week 7, respectively.

### DETAILED DESCRIPTION

The invention generally relates to methods for the prevention and treatment of adverse skin reactions resulting from radiation therapy. These methods generally include applying to a topical site a hydrogel composition including one or more electrolytes. The invention further provides hydrogel compositions that are useful for the practice of the methods of the invention.

Throughout the description, where compositions are described as having, including, or comprising specific components, or where processes are described as having, including, or comprising specific process steps, it is contemplated that compositions of the present invention also consist essentially of, or consist of, the recited components, and that the processes of the present invention also consist essentially of, or consist of, the recited processing steps.

Additionally, as used herein, the singular forms “a,” “an,” and “the” refer to “one or more” unless the context clearly dictates otherwise. Thus, for example, reference to “a protein” refers not only to a single protein but also to two or more proteins such as a mixture of proteins, and “a polymer” refers not only to one type of polymer but a plurality of polymers such as a blend of polymers and the like. That is, use of the singular includes the plural (and vice versa) unless specifically stated otherwise. In addition, where the use of the term “about” is before a quantitative value, the present teachings also include the specific quantitative value itself (and vice versa) unless specifically stated otherwise.

It also should be understood that the order of steps or order for performing certain actions is immaterial so long as the invention remains operable. Moreover, two or more steps or actions may be conducted simultaneously.

About 80% of patients receiving radiation therapy for breast cancer develop radiation dermatitis. Both acute and chronic skin damage can be caused by exposure to ionizing radiation. As used herein, “ionizing radiation” refers to radiation commonly employed in the treatment of tumors (whether benign or cancerous) which, either as a large single dosage or as repeated smaller dosages, will cause acute and/or late skin damage in at least a portion of the patients exposed to this dosage of radiation. Ionizing radiation includes, by way of example, x-rays, electron beams, gamma rays (γ-rays), and the like. The length and dosage of radiation treatment vary greatly from patient to patient, and are governed by a variety of factors, such as the origin of the cancer, the location and size of the tumor, the general health of the patient, and so forth. The treatment regime also usually is coordinated with any conjunctive treatment the patient may be receiving. Typical radiation doses are in the range of about 1 Gy to about 80 Gy.

As used herein, “acute radiation-induced skin damage” refers to damage to the epidermal layer caused by either a single large dosage or repeated smaller dosages of ionizing radiation. Such damage can manifest itself about 3-5 weeks after treatment with ionizing radiation. Acute radiation-induced skin damage can include, by way of example, erythema, edema, hyperpigmentation, dry desquamation, moist desquamation, epilation and ulceration. Acute radiation-induced skin damage can be particularly severe in skin folds and areas of high friction, e.g., the groin, the folds of the breast, the neck, behind the ears, and the like.

As used herein, “erythema” refers to abnormal redness of the skin caused by dilation and congestion of the capillaries, e.g., as a result of inflammation, injury, or burn.

As used herein, “edema” refers to swelling of the skin caused by excessive accumulation of serous fluid in the interstitial space of the epidermis.

As used herein, “dry desquamation” refers to shedding or peeling of epidermis in scales. Dry desquamation is an acute phenomenon that correlates with the depletion of actively proliferating basal cells in the epidermal layer; specifically, a fixed percentage of basal cells die with each dose fraction of ionizing radiation. Remaining basal cells undergo cornification and shed at an increased rate, leading to peeling of epidermis.

As used herein, “moist desquamation” refers to loss of epidermis caused by sterilization of a high proportion of cells within the basal layer of the epidermis.

As used herein, “late radiation-induced skin damage” refers to skin damage arising 6 or more months after termination of the radiation treatment which damage can include, by way of example, atrophy, fibrosis, thinning of the skin, telangiectasia, altered pigmentation, ulceration, necrosis and carcinogenesis.

As used herein, “adverse skin reactions” and “radiation dermatitis” can include both acute radiation-induced skin damage and late radiation-induced skin damage. In addition, both conditions emcompass any cutaneous and subcutaneous inflammatory reactions or reactions of skin toxicity occurring as a result of exposure to ionizing radiation.

As used herein, a method for preventing adverse skin reactions can include any procedure performed or application of product to maintain the integrity and function of the skin. This includes procedures performed or products applied to avoid and/or reduce any physical and/or chemical impacts that can affect skin homeostatis. Prophylaxis of adverse skin reactions also can be viewed as any possible measures taken promptly to recover skin integrity and function after certain physical and/or chemical impacts have caused skin damage.

As used herein, a method for treating adverse skin reactions can include any procedure performed or application of product to restore the integrity and function of the skin after injury. Treatment of adverse skin reactions can be carried out by one or more of the following: shielding the injured site from further physical and/or chemical insult, protecting injured site from microbial contamination, supporting and acclerating the natural process of healing, and the like. Because of such treatment, the skin injury can heal faster, be reduced in degree (for example, symptoms of skin reactions can be alleviated to a tolerable degree such that radiation treatment need not be interrupted or can be resumed earlier if interruptions are necessary), or the onset of any skin damage can be delayed, for example, until after termination of radiation treatment.

In a first aspect, the invention provides a method of preventing one or more adverse skin reactions caused by radiation therapy. The method generally includes applying to a topical site a hydrogel composition including one or more electrolytes within four hours of a first exposure of the topical site to ionizing radiation. Examples of suitable electrolytes include, but are not limited to, sodium chloride (NaCl), sodium phosphate (Na₂HPO₄ and NaH₂PO₄), other sodium salts such as a sodium salt of ethylenediaminetetracetic acid (EDTA), various mineral salts e.g., potassium, magnesium, calcium, bicarbonates, and the like. As used herein, “electrolytes” can include any substance that dissociates into two or more ions when dissolved in water, including various acids, bases, or salts. The one or more electrolytes usually are water-soluble so that the hydrogel composition, when applied, can be hydrated and contain an effective amount of electrolyte(s). For example, the hydrogel composition can include a water content of 50% or more by weight, a water content of 60% or more weight, a water content of 70% or more by weight, a water content of 80% or more by weight, a water content of 85% or more by weight, a water content of 90% or more by weight, or a water content of 95% or more by weight. In addition, the hydrogel composition can include about 5-10% by weight of one or more electrolytes, about 2-5% by weight of one or more electrodes, or about 0.5-2% by weight of one or more electrolytes.

To prevent one or more skin reactions resulting from ionizing radiation, the hydrogel composition can applied immediately after a first exposure of the topical site to ionizing radiation. For example, the hydrogel composition can be applied within four hours of first exposure, within two hours of first exposure, within one hour of first exposure, within thirty minutes of first exposure, within fifteen minutes of first exposure, within ten minutes of first exposure, or within five minutes of first exposure. The hydrogel composition can be continuously applied on the topical site until the next exposure of the topical site to ionizing radiation, typically 24 hours after the first exposure. Because a typical radiation therapy regimen includes five weeks or more of daily exposure to ionizing radiation, a hydrogel composition can be applied daily for five or more consecutive weeks in between exposures to ionizing radiation. In some embodiments, the method includes daily application of a hydrogel composition to the topical site throughout the radiation treatment regimen and for two additional weeks after termination of radiation treatment.

Hydrogel compositions suitable for the practice of the methods of the invention generally include a protein component and a biocompatible polymer component. The biocompatible polymer component of the hydrogel compositions can include various homopolymers, copolymers, or blends of polymers, all of which are biocompatible. As used herein, “biocompatible polymer” refers to a natural or synthetic polymer which, alone or in combination with other biocompatible polymers, can form a water-insoluble polymeric layer over the skin that is compatible with the skin as measured by the lack of skin irritation and can be removed from the skin by conventional means, preferably atraumatically. Examples of suitable bicompatible polymers include polyalkylene oxides, polymethacrylates, polyurethanes, cellulosics, polyhydroxyalkyl acrlaytes, polyesters, and the like.

In certain embodiments, the biocompatible polymer component of the hydrogel compositions includes at least one hydrophilic polymer capable of incorporating and binding relatively high concentrations of water. Examples of such polymers include, but are not limited to, polyethylene glycol (PEG) and its derivatives, for example, various polyethylene glycols having reactive terminal groups (e.g., carbonates of PEG) or substituents covalently attached to the ethylene carbon atoms of the molecule. When reference to polyethylene glycol or PEG is made herein, it includes such derivatives unless specifically reciting an underivatized PEG. The biocompatible polymer component should have sufficient molecular weight such that if reacted with a protein component, it readily crosslinks the protein component and the composition gels within a relatively short time. Generally, polymers with weight average molecular weights in the range of about 0.05 Da to about 10×10⁴ Da, or about 0.2 Da to about 3.5×10⁴ Da, or about 8,000 Da are employed.

Examples of suitable hydrogel compositions are described in U.S. Pat. No. 5,733,563, International Application Publication No. WO 01/74928, and U.S. patent application Ser. No. 10/970,349. The protein component can be crosslinked by the biocompatible polymer component. The protein component can be obtained from a variety of sources including vegetal sources (e.g., soybean or wheat), animal sources (e.g., milk, egg, or bovine serum), and marine sources (e.g., fish protein or algae). Suitable proteins include, but are not limited to, bovine serum albumin, human serum albumin, lactalbumin, ovalbumin, soy albumin, pea albumin, hydrolyzed soy protein, hydrolyzed wheat protein, casein, and any of their combinations. Proteins with abundant charge groups are preferred since they confer excellent water-retaining capacity to the hydrogel compositions.

The hydrogel compositions can further include buffering agents, antimicrobial agents, and other additives, including colorants, fragrance, binders, plasticizers, stabilizers, fire retardants, cosmetics, and moisturizers. Suitable buffering agents, antimicrobial agents, and various additives are known by those skilled in the art and are described in co-owned, co-pending U.S. patent application Ser. No. 10/970,349.

To facilitate application on a topical site, the hydrogel compositions can include a backing or support. The backing can be polymeric and can be attached to the hydrogel composition with or without the use of an adhesive. As disclosed in co-owned, co-pending U.S. patent application Ser. No. 10/471,463, a polymeric backing can be adhered to the hydrogel composition by exposing the surface of the polymeric backing to an activated gas. More specifically, a polymeric backing, such as polyethylene terephthalate, can be exposed to plasma of various gases or mixture of gases, including, but not limited to, nitrogen, ammonia, oxygen, and various noble gases, produced by an excitation source such as microwave and radiofrequency. A polymeric backing so treated typically adheres to a hydrogel composition according to the invention.

In preferred embodiments, the hydrogel composition is fully hydrated when applied to the topical site. A fully hydrated hydrogel composition can maximize the hydrating effect of the hydrogel composition, which helps to relieve tenderness, burning, itch, pain, and other undesirable sensations that can accompany radiation-induced skin reactions. Without wishing to be bound to any particular theory, this hydrating effect also is believed to be effective in limiting skin damage potentially caused by irradiation of the skin. To prevent water evaporation, the hydrogel composition can include an occlusive membrane, which can be or include the backing or support. Preferably, the occlusive membrane is oxygen-permeable.

Application of the hydrogel composition to a topical site can produce a cooling sensation to the patient. Although this sensation can induce a soothing effect for some patients and therefore can be desirable, some patients may find it too chilling and uncomfortable. To minimize such a chilling sensation, a mesh can be embedded within the hydrogel composition. Without wishing to be bound to any particular theory, such a mesh is believed to control, e.g., reduce, the rate of evaporation of water from the hydrogel composition and the topical site. In certain embodiments, the backing of the hydrogel composition contains perforations such as holes or slits to control the rate of evaporation.

To prevent unnecessary damage to the topical site, a hydrogel composition can be non-adherent to the topical site to be irradiated and/or non-adhesive to the surrounding areas of the topical site. Accordingly, the hydrogel composition can be described as atraumatic. It was previously demonstrated in U.S. Pat. No. 5,733,563 that hydrogel compositions can possess good mechanical properties. Particularly, the hydrogel composition can conform to the contours of the topical site both during motion and at rest, which can minimize disruption to the daily activities of the patient. To secure the hydrogel composition to the topical site, flexible netting tubes, such as Netelast (Seton Healthcare Group Plc, Oldham, UK), can be used.

In some embodiments, the hydrogel composition can be securely attached to an inner side (i.e. the side having contact with a patient's body) of a piece of a garment, e.g., an undergarment. More specifically, a surface of the hydrogel composition or its backing can be attached to a layer of pressure-sensitive adhesive, e.g., a biocompatible silicone. The layer of pressure-sensitive adhesive can then be applied to the inner side of a piece of a garment, e.g., a bra, thereby securing the hydrogel composition to the topical site without risk of causing trauma due to use of secondary dressings that can adhere to the topical site or surrounding areas. In certain embodiments, the layer of pressure-sensitive adhesive can first be applied to the inner side of the piece of the garment. The hydrogel composition can then be deposited on top of the adhesive layer.

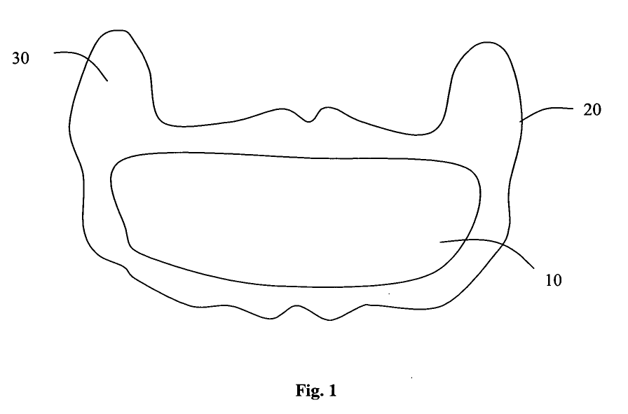

FIG. 1 shows a hydrogel composition 10 attached to a layer of pressure-sensitive adhesive 20. The pressure-sensitive adhesive layer can be coated on one or both surfaces of a porous support 30. On the surface that is to be applied adjacent to the skin, the porous support can include adhesives only in the area where the hydrogel composition is to be attached. Additionally, the porous support and the adhesive layer can be cut to a shape that facilitates secure attachment to the garment as shown in FIG. 1. Also, release liners can be used to protect the adhesive layer prior to its attachment to the garment and/or hydrogel composition. Other means can be used to secure the hydrogel composition to the topical site, as long as the securing means can be removed, preferably with little or no trauma to the topical site.

The hydrogel composition can be applied to any topical site that can be treated with ionizing radiation. This includes anatomical areas including, but not limited to, the cervical area, the breast area, the axilla, the abdominal area, the cranial area, the thoracic area, the inguinal area, the perianal area, and the perineum area.

A second aspect of the invention relates to a method of treating one or more adverse skin reactions resulting from ionizing radiation. Such method of treatment can include reducing the degree or accelerating the healing of any skin injury, delaying the onset of adverse skin reactions, and/or alleviating the symptoms of such skin reactions, that can result from exposure to ionizing radiation. The method includes applying to a topical site an embodiment of the hydrogel compositions described above after exposure of the topical site to ionizing radiation: The topical site can be intact skin or an open wound displaying symptoms of one or more adverse skin reactions. In certain embodiments, the method includes daily application of a hydrogel composition to the topical site until all physical signs of skin injury disappear.

A third aspect of the invention relates to methods of preparing a hydrogel composition. The method generally includes reacting a protein component with a bifunctional biocompatible polymer. The protein component can include any of the proteins already described. The bifunctional biocompatible polymer can be a bifunctional polyethylene glycol, such as a dinitrophenylcarbonyl polyethylene glycol, a dichlorosulfonyl polyethylene glycol, a dichloroacetylsulfonyl polyethylene glycol, a dichlorosulfonyl ethylsulfonyl polyethylene glycol, a diphenylcarbonyl polyethylene glycol, a ditoluenesulfonyl polyethylene glycol, a disuccinimidyl polyethylene glycol, a dimaleimidyl polyethylene glycol, a diisocyanato-polyethylene glycol, or a divinylsulfonamido-polyethylene glycol. International Application Publication No. WO 01/74928 discloses that bifunctional polyethylene glycols such as the ones listed above can be used to form hydrogel compositions by mixing the bifunctional polyethylene glycol with proteins dissolved in aqueous solutions under basic conditions.

The method of preparing a hydrogel composition can include converting a biocompatible polymer into a bifunctional biocompatible polymer. As described in co-owned, co-pending U.S. patent application Ser. No. 10/970,349, to effect covalent attachment of a PEG to a protein, the hydroxyl end-groups of the polymer can be first converted into reactive functional groups. This process is frequently referred to as “activation” and the resulting bifunctional polyethylene glycol can be described by formula 1:
X—O—(CH₂CH₂O)ₙ—X (1)
where X can be any functional group able to react with the various chemical groups commonly found in proteins, including amino, thiol, hydroxyl, carboxyl and carboxylic groups; and n can vary from about 45 to about 800, which corresponds to commercial PEG of molecular weight ranging from about 2,000 to about 35,000 Daltons. The activation step can be conducted in solvent or in a solvent-free environment as detailed in co-owned, co-pending U.S. patent application Ser. Nos. 10/487,392 and 11/071,877.

U.S. patent application Ser. No. 10/487,392 describes a method for preparing activated PEGs with p-nitrophenyl chloroformate. The method involves a reaction carried out at room temperature using an aprotic solvent, such as methylene chloride (CH₂Cl₂), in the presence of a catalyst, such as dimethylaminopyridine (DMAP). U.S. patent application Ser. No. 11/071,877 describes alternative methods of activating PEG, e.g., by reacting molten PEG with an activator in a solvent-free environment. Additionally, commercial PEG-dinitrophenyl carbonates suitable for preparing hydrogel compositions of the present invention are available, and can be purchased from Nektar Therapeutics (Huntsville, Ala.).

Hydrogel compositions can have a variety of desirable properties. Their hydrating effect on topical sites, both short-term and long-term, was demonstrated in co-owned co-pending U.S. patent application Ser. No. 10/970,349. As described in co-owned, co-pending U.S. patent application Ser. Nos. 10/970,349 and ______ [Attorney Docket No. BAG-012], it also has been shown that hydrogel compositions can accelerate the healing of various types of wounds, and can reduce topical inflammatory response without inducing toxicity or causing irritation to human skin.

The following examples are provided to illustrate further and to facilitate the understanding of the invention and are not intended to limit the scope of the invention in any way.

### EXAMPLE 1

### Preparation of an Embodiment of a Hydrogel Composition

An aqueous solution of activated polyethylene glycol (PEG) was mixed with an equal volume of a soy protein solution. The resultant mixture was cast between two films to give a hydrogel with a thickness of about 1.8 mm and cut to a dimension of about 8 cm by 20 cm. After polymerization, the hydrogel was incubated in a buffered solution to remove by-products and unreacted PEG and soy protein. The purified hydrogel was submerged in a phosphate-buffered saline solution containing ethylenediaminetetraacetic acid (EDTA) (0.9 wt. % sodium chloride, 0.2 wt. % EDTA, and 0.16 wt. % sodium phosphate monobasic) and preservatives at pH 5.5 to integrate one or more electrolytes into the hydrogel.

### EXAMPLE 2

### Study on Human Subjects Receiving Radiation Therapy as Treatment for Breast Cancer

Fifteen patients with breast cancer receiving radiation therapy were equally divided into three groups and randomized to receive one of the following treatments: (1) a preventive treatment with the hydrogel composition of Example 1, (2) a curative treatment with the hydrogel composition of Example 1, and (3) a curative treatment with a commercially available trolamine ointment. The efficacy of the hydrogel composition for the prevention and intervention of radiation-induced skin toxicity was evaluated.

Skin toxicity grading has previously been defined by the Radiation Therapy Oncology Group (RTOG). Grade 0 skin toxicity corresponds to no physical signs of skin toxicity. Grade 1 skin toxicity displays follicular, faint, or dull erythema; epilation (loss of hair), dry desquamation, or decrease in sweating. Grade 2 skin toxicity corresponds to tenderness with bright erythema; patchy, moist desquamation or moderate edema. Grade 3 skin toxicity is defined as having confluent, moist desquamation, other than skin folds, and pitting edema. Grade 4 skin toxicity exhibits ulceration, hemorrhage, and necrosis. Secondary measures included weekly assessments of pain and patient satisfaction.

Prophylactic treatment with hydrogel compositions was initiated immediately after first radiation treatment and continued for two weeks after termination of radiation, while curative treatment with hydrogel compositions was initiated at the first sign of radiation-induced skin toxicity (typically after three weeks of radiation therapy) and continued for two weeks after termination of radiation. In either case, a hydrogel composition was applied to the skinfold area underneath the irradiated breast immediately (i.e., within 5 minutes) after radiation exposure. The hydrogel composition was removed prior to the next radiation treatment and a new hydrogel composition was applied immediately after. During weekends and after termination of the radiation regimen, a new hydrogel was applied after 24 hours of application.

Patients receiving curative treatment with the trolamine ointment were instructed to apply the product twice a day, seven days a week, after the first sign of skin toxicity was observed and continued for two weeks after termination of radiation treatment. One of the two daily applications was done immediately following the daily radiation treatment. The patients were instructed not to apply the product less than four hours prior to the daily radiation treatment. The treatment area was gently cleansed prior to each application to prevent product build up.

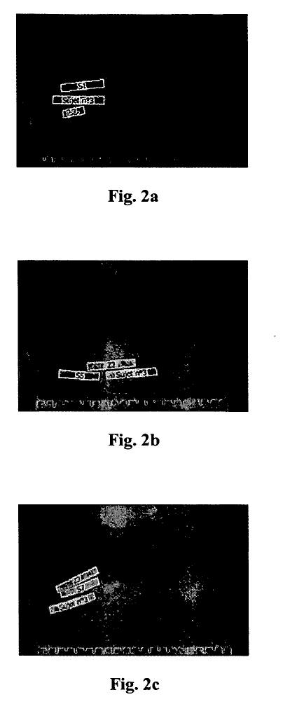

Three of the five patients receiving preventive treatment with hydrogel compositions reported no experience of pain, itching or burning in the irradiated area. Also, referring to FIGS. 2a-c, these patients exhibited no physical sign of skin toxicity throughout and two weeks after termination of the radiation regimen. Two of the patients did display symptoms of Grade 1-2 radiation dermatitis between two to three weeks after the first radiation exposure. More specifically, both patients developed 2-3 skin lesions, each of which resembled small red bumps and had a diameter of less than 5 mm. All five patients receiving preventive treatment with hydrogel compositions were able to complete the regimen without interruption.

Patients who received curative treatment with hydrogel compositions displayed symptoms of Grade 1-2 radiation dermatitis between two to three weeks after the first radiation exposure. Three patients left the study for personal reasons before completion of the study. With respect to the two remaining patients, all physical signs of skin toxicity were reported to have disappeared after 7-11 days of daily treatment with hydrogel compositions. Patients also reported a refreshing sensation and a cooling effect with the use of the hydrogel composition which alleviated any pain or soreness they might have experienced in the irradiated area.

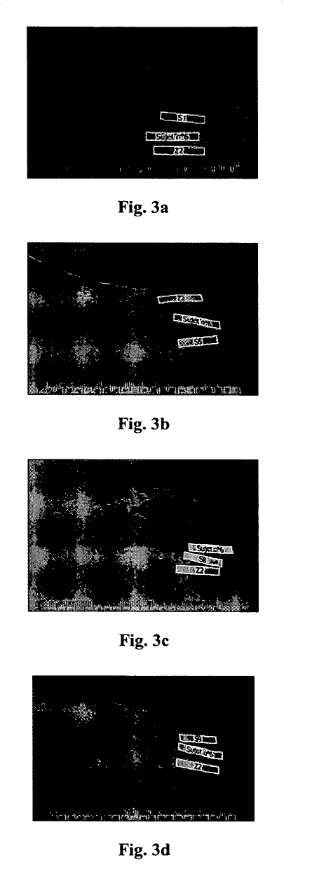

FIGS. 3a-d show the evolving skin reactions experienced by one of the two remaining patients after eight weeks of radiation therapy. FIG. 3c shows an open wound in the skinfold area underneath the irradiated breast at week 8. The open wound was observed to have healed after one week of daily treatment with hydrogel compositions as shown in FIG. 3d.

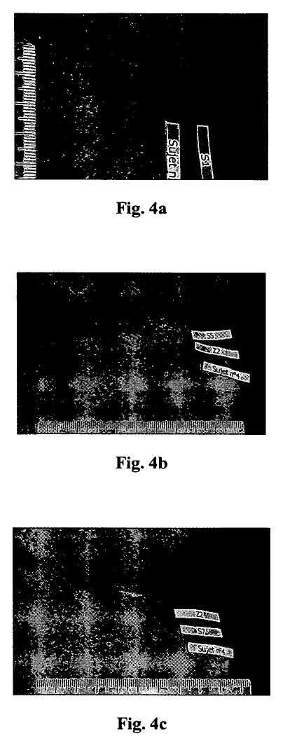

Among the group of patients which received curative treatment with the trolamine ointment, all five patients displayed symptoms of Grade 1-2 radiation dermatitis between two to three weeks after the first radiation exposure. More specifically, two patients developed mild erythema, one patient developed severe erythema, and open wounds were observed on the other two patients at week 5. Treatment with the trolamine ointment did not seem to have improved the skin damage for these patients. Patients receiving this treatment also reported that it was painful to apply the ointment. FIGS. 4a-c show the evolving skin condition of the patient developing severe erythema at week 5 (FIG. 4b). As can be seen in FIG. 4c, the erythema did not subside and dry desquamation was observed at week 7 despite treatment with the trolamine ointment.

Variations, modifications, and other implementations of what is described herein will occur to those of ordinary skill in the art without departing from the spirit and the essential characteristics of the invention. Accordingly, the scope of the invention is to be defined not by the preceding illustrative description but instead by the following claims, and all changes that come within the meaning and range of equivalency of the claims are intended to be embraced therein.

Each of the patent documents and scientific publications disclosed hereinabove is incorporated by reference herein for all purposes.

What is claimed is:

1. A method of preventing one or more adverse skin reactions resulting from ionizing radiation, the method comprising:
applying a hydrogel composition comprising one or more electrolytes to a topical site within four hours of first exposure of the topical site to ionizing radiation.

2. The method of claim 1, wherein the one or more electrolytes are selected from a sodium salt of ethylenediaminetetraacetic acid, sodium chloride, and sodium phosphate.

3. The method of claim 1, wherein the hydrogel composition comprises a biocompatible polymer.

4. The method of claim 3, wherein the hydrogel composition comprises a protein component crosslinked by the biocompatible polymer.

5. The method of claim 4, wherein the protein component comprises one or more proteins selected from the group consisting of bovine serum albumin, human serum albumin, lactalbumin, ovalbumin, soy albumin, pea albumin, hydrolyzed soy protein, hydrolyzed wheat protein, casein, and combinations thereof.

6. The method of claim 3, wherein the biocompatible polymer comprises polyethylene glycol or a derivative thereof.

7. The method of claim 1, wherein the hydrogel composition is applied to the topical site within thirty minutes of a first exposure of the topical site to ionizing radiation.

8. The method of claim 1, wherein the topical site comprises intact skin.

9. The method of claim 1, wherein the method prevents at least one type of adverse skin reaction that is symptomatic of acute or chronic radiation dermatitis.

10. The method of claim 1, wherein the method prevents at least one type of adverse skin reaction selected from erythema, hyperpigmentation, epilation, edema, dry desquamation, moist desquamation, ulceration, hemorrhage, and necrosis.

11. A method of treating, reducing, and/or delaying one or more adverse skin reactions resulting from ionizing radiation, the method comprising:
applying a hydrogel composition comprising one or more electrolytes to a topical site after exposure of the topical site to ionizing radiation.

12. The method of claim 11, wherein the one or more electrolytes are selected from a sodium salt of ethylenediaminetetraacetic acid, sodium chloride, and sodium phosphate.

13. The method of claim 11, wherein the hydrogel composition comprises a biocompatible polymer.

14. The method of claim 13, wherein the hydrogel composition comprises a protein component crosslinked by the biocompatible polymer.

15. The method of claim 14, wherein the protein component comprises one or more proteins selected from the group consisting of bovine serum albumin, human serum albumin, lactalbumin, ovalbumin, soy albumin, pea albumin, hydrolyzed soy protein, hydrolyzed wheat protein, casein, and combinations thereof.

16. The method of claim 13, wherein the biocompatible polymer comprises polyethylene glycol or a derivative thereof.

17. The method of claim 11, wherein the topical site comprises intact skin or a wound.

18. The method of claim 11, wherein the topical site is located in a cervical area, a breast area, an abdominal area, a cranial area, a thoracic area, an inguinal area, a perianal area, or a perineum area.

19. The method of claim 11, wherein the hydrogel composition is applied to the topical site for at least 12 consecutive hours.

20. The method of claim 11, wherein the method comprises applying daily for at least seven consecutive days a hydrogel composition comprising one or more electrolytes to the topical site after exposure of the topical site to ionizing radiation.
